(19) United States
(12) Patent Application Publication
(10) Pub. No.: US 2024/0319185 A1
(43) Pub. Date: Sep. 26, 2024

(54) DEVELOPMENT OF NOVEL APTAMER-BASED BIOSENSOR FOR DETECTION OF SARS-COV-2 PROTEIN IN NASOPHARYNGEAL SWABS OF COVID-19 PATIENTS

(71) Applicant: University of Sharjah, Sharjah (AE)

(72) Inventors: Rabih HALWANI, Sharjah (AE); Fatemeh Saheb SHARIF-ASKARI, Sharjah (AE); Qutayba HAMID, Sharjah (AE); Zainab AL SHAREEF, Sharjah (AE); Rifat HAMOUDI, Sharjah (AE)

(21) Appl. No.: 18/126,108

(22) Filed: Mar. 24, 2023

(51) Int. Cl. G01N 33/569; G01N 21/64; G01N 33/58

(52) U.S. Cl. CPC G01N 33/56983; G01N 21/6486; G01N 33/582; G01N 2333/165

(57) ABSTRACT

An aptamer-based biosensor for detecting SARS-COV-2 in symptomatic and asymptomatic patients capable of onsite testing of large-scale population of up to 96 people at once providing fast and reliable results. The present invention relates to a novel aptamer-based biosensor for the rapid and reliable detection of SARS-COV-2 viral proteins in nasopharyngeal swabs of suspected individuals with COVID-19.

### TECHNICAL FIELD

The present invention relates to a novel aptamer-based biosensor for the rapid and reliable detection of SARS-COV-2 viral proteins in nasopharyngeal swabs of suspected individuals with COVID-19.

### REFERENCE TO AN ELECTRONIC SEQUENCE LISTING

The contents of the electronic sequence listing (sequencelisting.xml; Size: 4,096 bytes; and Date of Creation: Nov. 16, 2023) is herein incorporated by reference in its entirety.

### BACKGROUND

COVID-19 caused by severe acute respiratory syndrome coronavirus 2 (SARS-COV-2) infection emerged more than a year ago, but it still keeps a strong grip on diagnostic capacities.

Currently, only the RT-qPCR is routinely used to detect SARS-COV-2 infection. However, the test takes time (at least 48 hours), energy and trained personnel to run. It is also costly and thus limit large-scale population testing. Moreover, other drawback of RT-qPCR is that it lacks specificity; negative tests need to be interpreted with caution, taking into account pre-test probability of COVID-19 like illness of tested individuals (Yoshida et al., Anal. Bioanal. Chem. 395:1089-1096, 2009). Rapid antigen detection tests (Ag RDT) for SARS-COV-2 appeared on the market in early 2020, but initial reports of poor performance and the lack of independent evaluation results made governments reluctant to invest and consider inclusion into testing algorithms.

Aptamer-based biosensors (aptasensors) have been designed and implicated to detect different kinds of viruses. For example, several reports have been published on the successful applications of Norovirus or Salmonella aptasensors in a multiple food analytical assay (Weng and Neethirajan, Microchimica Acta. 184:4545-4552, 2017; and Zou et al., Front. Microbiol. 10, 2019). Additionally, during the last outbreaks of H5N1 influenza in animal and human “bird flu”, aptasensors for detection of avian influenza virus (AIV) H5N1 in poultry swab samples was developed (Bai et al., Sensors. 12, 2012). There are also reports about successful detection of HIV-1, Dengue, RSV using fluorescence-conjugated aptamers in different clinical settings (Weng and Neethirajan, Microchimica Acta. 184:4545-4552, 2017). Till now, no such test exists for detection of SARS-COV-2. Therefore, there is an urgent need for developing a reliable, sensitive, and rapid COVID-19 diagnostic test with improved specificity; this necessity detection of SARS-COV2 protein rather than the virus mRNA levels.

### SUMMARY

In accordance with the present invention, there is provided an aptamer-based biosensor for the rapid and reliable detection of SARS-COV-2 in the nasopharyngeal swabs of COVID-19 suspected individuals. The aptamer-based biosensor can be utilized for onsite screening for COVID-19 infections and is capable of testing 96 samples at once and provide test results in approximately few minutes.

In a first aspect, a biosensor for detecting SARS-COV-2 includes an aptamer that can bind a protein, a fluorescent dye conjugated to the aptamer, and a graphene oxide solution.

In a most preferred aspect, the aptamer includes the nucleotide sequence of SEQ ID NO: 1.

In another aspect, a fluorescent substance is conjugated to the nucleotide sequence of the aptamer.

In another aspect, the fluorescent substance includes a fluorophore and the fluorophore includes a 6-carboxyfluorescein (6-FAM).

In another aspect, the biosensor includes a mixture of an approximately equal volume of graphene oxide and 6-carboxyfluorescein conjugated aptamer.

In a preferred aspect, the aptamer can bind to antigens including the SARS-COV-2 virus.

In a most preferred aspect, the antigen includes the SARS-COV-2 protein S.

In another aspect, the SARS-COV-2 protein S is detected in samples from subjects with symptomatic or asymptomatic COVID-19.

In a preferred aspect, the subjects are human.

In a most preferred aspect, the human subjects are patients who may present symptomatic or asymptomatic COVID-19.

In some aspects, the patients may not have COVID-19.

In another aspect, the samples from patients are collected from nasopharyngeal swabs or from saliva samples.

In a preferred aspect, the samples from patients are collected from nasopharyngeal swabs.

In another aspect, the samples are analyzed, and the results are collected.

In a second aspect of the present invention, there is provided an aptamer-based method for detecting SARS-COV-2, comprising forming a biosensor, where the biosensor comprises an aptamer, a fluorescent substance conjugated to the aptamer, and graphene oxide.

In a preferred aspect, the aptamer-based method includes a mixture of an approximately equal volume of graphene oxide and 6-carboxyfluorescein conjugated aptamer that is added to a detection area on a glass-based reading substrate.

In some aspects of the aptamer-based method, the fluorescent substance conjugated to the aptamer comprises a fluorophore.

In a preferred aspect of the aptamer-based method, the fluorophore comprises a 6-carboxyfluorescein.

In a most preferred aspect of the aptamer-based method, the aptamer includes the nucleotide sequence of SEQ ID NO: 1.

In a preferred aspect of the aptamer-based method, the aptamer can bind to antigens including the SARS-COV-2 virus.

In a most preferred aspect of the aptamer-based method, the antigen includes the SARS-COV-2 protein S.

In some aspects of the aptamer-based method, the SARS-COV-2 protein S is detected in samples from subjects with symptomatic or asymptomatic COVID-19.

In some aspects of the aptamer-based method, the subjects are human.

In some aspects of the aptamer-based method, the human subjects are patients who may present symptomatic or asymptomatic COVID-19.

In some aspects of the aptamer-based method, the patients may not have COVID-19.

In some aspects of the aptamer-based method, the samples from patients are collected from nasopharyngeal swabs or from saliva samples.

In a preferred aspect of the aptamer-based method, the samples from patients are collected from nasopharyngeal swabs.

In some aspects of the aptamer-based method, the samples are analyzed and the results are collected.

The term “subject” in accordance with the present invention, includes, e.g., mammals, such as dogs, cats, horses, rats, mice, monkeys, and humans.

### BRIEF DESCRIPTION OF THE DRAWINGS

The invention can be better understood with reference to the following figures and description. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 provides a schematic of the higher recovery signals of RBD-aptamer with recombinant SARS-COV-2 S than with other non-specific proteins. The data shown are representative of 10 independent experiments.

FIG. 2 provides a schematic of the values of ROS-FC₁ₘᵢₙ and Ct of SARS-COV-2 nucleocapsid (N1) gene for the tested nasopharyngeal swabs. The data presented show that a higher level of ROS-FC₁ₘᵢₙ was noticed in nasopharyngeal swabs with high viral loads or SARS-COV-2 N1 gene expression level. Notably, the viral loads in these samples were determined by RT-PCR that is, the greater the viral loads, the lower the Ct value.

FIG. 3 provides a schematic of the correlation between ROS-FC₁ₘᵢₙ and Ct of SARS-COV-2 nucleocapsid (N1) gene in the tested nasopharyngeal swabs. The data presented show that a significant higher level of ROS-FC₁ₘᵢₙ was noticed in the nasopharyngeal swabs with high viral loads or SARS-COV-2 N1 gene expression levels. Notably, the viral loads in these samples were determined by RT-PCR that is, the greater the viral loads, the lower the Ct value.

FIG. 4 provides a schematic of the correlation between ROS-FC₁ₘᵢₙ and viral loads of SARS-CoV-2 for the tested nasopharyngeal swabs.

### DEFINITIONS

As used in this description and the accompanying claims, the following terms shall have the meanings indicated, unless the context otherwise requires:

As used in this disclosure, including the appended claims, the singular forms “a,” “an,” and “the” include plural references, unless the content clearly dictates otherwise.—and are used interchangeably with “at least one” and “one or more.” Thus, reference to “an aptamer” includes mixtures of aptamers, and the like.

To the extent that the term “include,” “have,” or the like is used in the description or the claims, such term is intended to be inclusive in a manner similar to the term “comprise” as “comprise” is interpreted when employed as a transitional word in a claim.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “protein” is to be given its respective ordinary and customary meanings to a person of ordinary skill in the art; the term is used without limitation to refer to a polymer of amino acids, or amino acid analogs, regardless of its size or function.

The term “aptamer” of the subject technology, further may include aptamers having a nucleotide sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the nucleotide sequence of the reference aptamer.

### DETAILED DESCRIPTION

The present disclosure, in some aspects, provides a DNA aptamer-based biosensor with detection capability for SARS-COV-2's spike (S) in the nasopharyngeal swabs of COVID-19 suspected individuals, of both symptomatic (high-viral loads) and asymptomatic COVID-19 (low-viral loads) individuals.

An “aptamer” is an oligonucleotide (RNA or DNA) that is developed to specifically bind and target specific proteins.

The present invention describes a fluorescently labeled-SARS-COV-2 specific aptamer mixed (ratio 1:1) with graphene oxide. Binding of the aptamer to graphene causes the sequestration of the fluorescent signal. When the purified SARS-COV-2's spike protein or a sample containing SARS-COV-2 virus is added to the detection “chip”, binding of the aptamer to the virus causes the de-sequestration (“recovery”) of the fluorescent signal (ROS) that will then be detected using a portable fluorescence detection devise.

The aptamer-based biosensor of the present invention, upon detecting the Spike protein of SARS-CoV-2 virus generate ROS from each sample that reach plateau in just 1 minute, hence giving rapid results, and depending on the reading plate, up to 96 testing can be performed at once, enabling large-scale population testing.

The aptamer-based biosensor described herein is capable of on-site testing of a large-scale population of up to 96 people at once, and the results can be obtained in as short as few minutes, providing an advantage over the standard method of SARS-COV-2 testing utilizing the RT-qPCR.

The aptamer-based biosensor has high affinity and specificity for the target proteins. It is well known that 1 mg of aptamer is sufficient for more than 10 million assays (Liu et al., Cell. 165:535-550, 2016). Therefore, the aptamer-based biosensor is suitable for commercial scale production enabling population-scale testing at low costs, which can contribute to controlling the spread of the COVID-19 virus.

In one aspect, a biosensor for detecting SARS-COV-2 includes a mixture of aptamer conjugated to a fluorescent dye and graphene oxide that can bind to S protein of SARS-COV-2.

In a most preferred aspect, the aptamer includes the nucleotide sequence of SEQ ID NO: 1.

In some examples, variations can be used in reference to the nucleotide sequence of the aptamer, where at least one of the four constituent nucleotide bases (i.e., A, G, T/U, and C) of the aptamer is an analog or ester of a naturally occurring nucleotide. In some cases, the modified nucleotide confers nuclease resistance to the oligonucleotide. A pyrimidin with a substitution at the C-position is an example of a modified nucleotide. Modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group mojeties of from about 1 to about 20 carbon atoms, polyethylene glycol (PEG) polymers in one embodiment ranging from about 10 to about 80 kDa, PEG polymers in another embodiment ranging from about 20 to about 60 kDa, or other hydrophilic or hydrophobic biological or synthetic polymers. In one embodiment, modifications are of the C-5 position of pyrimidines. These modifications can be produced through an amide linkage directly at the C-5 position or by other types of linkages.

In another aspect, a fluorescent substance is conjugated to the nucleotide sequence of the aptamer.

In some examples, at least one of a fluorescent substance, a nano luminescent material, biotin, digoxigenin, and an enzyme label is bound to a nucleic acid sequence of the aptamer of the present application.

In a preferred example, the fluorescent species is a fluorophore, and the fluorophore comprises a FAM fluorophore. It is understood that the FAM fluorophore is only one fluorophore employed in one implementation of the present application, and does exclude that other fluorophores are also possible.

In a most preferred aspect, the fluorescent substance includes a fluorophore and the fluorophore includes a 6-carboxyfluorescein (6-FAM).

In another aspect, the biosensor includes a mixture of an approximately equal volume of graphene oxide and 6-carboxyfluorescein conjugated aptamer.

In a preferred aspect, the aptamer can bind to antigens including the SARS-COV-2 virus.

In a most preferred aspect, the antigen includes the SARS-COV-2 protein S.

In another aspect, the SARS-COV-2 protein S is detected in samples from subjects with symptomatic or asymptomatic COVID-19.

In a preferred aspect, the subjects are human.

In a most preferred aspect, the human subjects are patients who may present symptomatic or asymptomatic COVID-19.

In some cases, the patients may not have COVID-19. However, the test is designed to rapidly detect SARS-COV-2 in the patients that are positive for COVID-19, especially in the asymptomatic population.

In another aspect, the samples from patients may be collected from nasopharyngeal swabs or from saliva samples.

In a preferred aspect, the samples from patients are collected from nasopharyngeal swabs.

In another aspect, the samples are analyzed and the results are collected.

In a second aspect of the present invention, there is provided an aptamer-based method for detecting SARS-COV-2, comprising forming a biosensor, where the biosensor comprises an aptamer, a fluorescent substance conjugated to the aptamer, and graphene oxide.

In a preferred aspect, the aptamer-based method includes a mixture of an approximately equal volume of graphene oxide and 6-carboxyfluorescein conjugated aptamer that is added to a detection area on a glass-based reading substrate.

In some aspects of the aptamer-based method, the fluorescent substance conjugated to the aptamer comprises a fluorophore.

In a preferred aspect of the aptamer-based method, the fluorophore comprises a 6-carboxyfluorescein.

In a most preferred aspect of the aptamer-based method, the aptamer includes the nucleotide sequence of SEQ ID NO: 1.

In some cases, the aptamer comprises a nucleotide sequence at least 70% (e.g., at least 70%, at least 75%, at least 80%, at least 85%, at least 90%, at least 99%, or at least 99%) identical to the nucleotide sequence of any one of SEQ ID NOs: 1.

In a preferred aspect of the aptamer-based method, the aptamer can bind to antigens including the SARS-COV-2 virus.

In a most preferred aspect of the aptamer-based method, the antigen includes the SARS-COV-2 protein S.

In some aspects of the aptamer-based method, the SARS-COV-2 protein S is detected in samples from subjects with symptomatic or asymptomatic COVID-19.

In some aspects of the aptamer-based method, the subjects are human.

In some aspects of the aptamer-based method, the human subjects are patients who may present symptomatic or asymptomatic COVID-19.

In some aspects of the aptamer-based method, the patients may not have COVID-19.

In some aspects of the aptamer-based method, the samples from patients are collected from nasopharyngeal swabs or from saliva samples.

In another aspect of the aptamer-based method, the samples are analyzed and the results are collected.

In some examples, the samples can be collected from swabs or fluid obtained from swabs, such as throat swabs, nasal swabs, nasopharyngeal swabs, nasal mid-turbinate swabs, oropharyngeal swabs, cheek swabs, saliva swabs, or other swabs, or other clinical samples.

The aptamer-based biosensor present invention offers an approach that is rapid, reliable, easy to manufacture, and relatively cost-effective. Therefore, it can be used for the quick mass screening of people coming through airports and border controls, educational institutes, hospitals, etc. For example, in an outbreak scenario, implementation of the aptamer-based biosensor into testing algorithms would enable rapid detection and isolation of contagious subjects, thus greatly facilitating the control of the spreading of the pandemic.

The optimal amounts of reagents for a particular reaction used in the biosensor of the present invention can be readily determined by those skilled in the art having the teachings herein.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### EXAMPLES

### Example 1—Preparation and Optimization of the Aptamer Sequence

To develop the proposed aptasensor, we first optimized the aptamer sequence of SARS-Spike (S)′ receptor binding domain (RBD-aptamer) in a way that it binds with high affinity to SARS-COV-2 S proteins. Additionally, these aptamers were FAM-fluorescence dye labelled. The FAM-conjugated RBD-aptamer was then synthesized by IDT company in USA and shipped to us. The 6-carboxyfluorescein (6-FAM) binding RBD aptamer was synthesized by Integrated DNA Technologies, Inc. (https://www.idtdna.com, Coralville, IA, USA) with the nucleotide sequence of SEQ ID NO: 1. (Song et al., Anal. Chem. 92:9895-9900, 2020).

### Example 2—Preparation of 6-FAM RBD-Aptamer Functionalized and/Graphene Oxide (GO); the “Chip” Assembly

For the preparation of the reading “chip” the working solution consisted of an equal volume of graphene oxide (2 mg/ml, Sigma Aldrich, Lot #MKCL918) mixed thoroughly with FAM-conjugated RBD-aptamer to quench the fluorescence of the aptamer (20 nM of aptamer in Nuclease-Free Water). The mixture was then pipetted onto the detection area (2 microliter of quenched-solution) of the glass-based reading chip, aptamer-based biosensor or “aptasensor” (Biotek Take3 Micro-Volume plate). The fluorescence signal intensity of the quenched-solution on the detection area was recorded as reference (I₀). The fluorescence spectra were measured at Ex/Em=485 nm/528 nm by the Multi-mode Reader. All samples were prepared in triplicate.

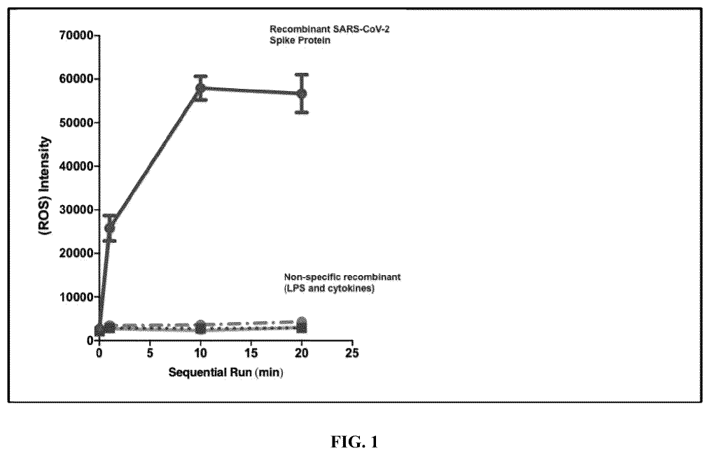

Having the reading “chip” ready, the aptamer-based biosensor's ability to detect SARS-COV-2 S viral proteins was then evaluated using specific S viral recombinant protein and non-specific random proteins. The aim was for aptamer-based biosensor to generate ROS following binding to S viral proteins but not from any other non-specific proteins. Here, the developed aptamer-based biosensor had significantly higher ROS with S protein compared to other non-specific proteins, indicating their specific binding to these viral proteins (FIG. 1).

During an assay, the ratio of change (fold change; FC) between the generated ROS at 1 minute after sample addition (ROS-FC₁ₘᵢₙ) and after quenching (I₀) was used to determine the SARS-COV-2 concentration in the sample. Here, the developed aptamer-based biosensor had significantly higher ROS-FC₁ₘᵢₙ with spike protein compared to other non-specific proteins (more than 50 ROS-FC₁ₘᵢₙ, respectively), indicating their specificity to SARS-COV-2 (FIG. 1).

### Example 2—Clinical Evaluation of the Developed “Aptasensor” for SARS-COV-2 Detection

The aptamer-based biosensor's ability to detect SARS-COV-2 was evaluated using COVID-19 positive and healthy control nasopharyngeal swabs. The ROS intensity generated from these swabs was compared to the result of RT-qPCR, the “gold standard” test for COVID-19.

### 1. Characteristic of the Tested Populations

During the study period of October 2020, to July 2021, a total of 375 suspected COVID-19 individuals were included in the study. Among the participants manifesting COVID-19 related-symptoms, 9.9% had severe and 23.9% had mild to moderate COVID-19 infection. Moreover, 25.1% had asymptomatic COVID-19 infection and 41.2% were negative for SARS-COV-2 by RT-qPCR (Table 1).

TABLE 1
Characteristics of the population of study

| Characteristics | Value, no. (%) | |
|---|---|---|
| Total | 375 | |
| *SARS-CoV-2 positive swabs | 275 | (58.8) |
| *SARS-CoV-2 negative swabs | 100 | (41.2) |
| #Symptoms reported | 82 | (33.8%) |
| Mild to moderate | 58 | (23.9) |
| Severe | 24 | (9.9) |
| #Asymptomatic | 61 | (25.1) |
| Ct N1 gene, median (range) | 29.72 | (24.85 |
| Ct > 40 (Negative) | 100 | (32.2) |
| Ct 39.9-20 | 67 | (21.5) |
| Ct < 20 | 73 | (23.5) |

*SARS-CoV-2 positivity and negativity were determined by qRT-PCR method.

### 2. Performance of the Developed RBD-Aptasensor

Out of 275 nasopharyngeal swabs that tested positive by RT-qPCR (Ct<40), 265 swabs were tested positive by RBD-aptasensor, defined as ROS more than 5 folds at 1 minute (ROS-FC₁ₘᵢₙ≥5) of sample addition (Table 2).

TABLE 2
Overview of results of comparison of RBD-
aptasensor and RT-qPCR for SARS-CoV-2

| | RT-qPCR result | | |
|---|---|---|---|
| RBD-aptasensor result | Positive# | Negative | Total |
| Positive* | 265 | 0 | 265 |
| Negative | 10 | 100 | 110 |
| Total | 275 | 100 | 375 |

*Positive result defined as ROS-FC_{1 min} ≥5.
#Positive result defined as Ct < 40 of N1 gene of SARS-CoV-2.

The performance of the aptamer-based biosensor when analyzed at ROS-FC₁ₘᵢₙ≥5 cutoff was of 96% sensitivity with 100% specificity (Table 2). Of note, the ROS-FC₁ₘᵢₙ≥5 cutoff was selected as reference as all the negative samples by RT-qPCR (Ct≥40) showed less than 5 folds at 1 minute. Moreover, while lower ROS-FC₁ₘᵢₙ≥5 cutoffs improve sensitivity it reduces the specificity of the aptamer-based biosensor considerably (Table 3).

TABLE 3
Performance of RBD-aptasensor stratified by various ROS-FC_{1 min} intervals

| | No. out of positive | | No. out of negative | |
|---|---|---|---|---|
| ROS-FC_{1 min} Intervals | RT-qPCR (n = 275)* | Sensitivity | RT- qPCR (n = 100)# | Specificity |
| FC >7 | 242/275 | 87% | 100/100 | 100% |
| FC >6 | 249/275 | 90% | 100/100 | 100% |
| FC >5 | 265/275 | 96% | 100/100 | 100% |
| FC >4 | 268/275 | 97.5% | 92/100 | 92% |
| FC >3 | 272/275 | 98.9% | 74/100 | 74% |
| FC >2 | 274/275 | 99.6% | 62/100 | 62% |

*Positive result defined as Ct < 40 of N1 gene of SARS-CoV-2.
#Negative result defined as Ct ≥ 40 of N1 gene of SARS-CoV-2.

3. Association of RBD-Aptasensor Results with RT-qPCR for SARS-COV-2

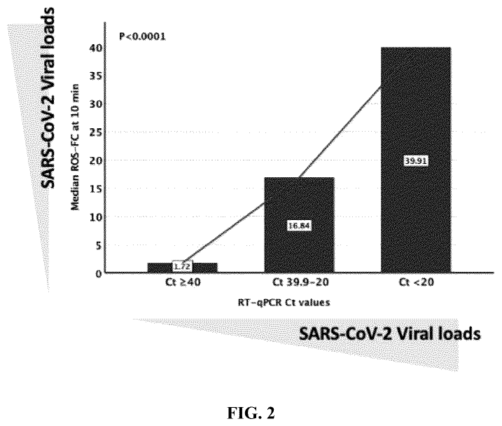

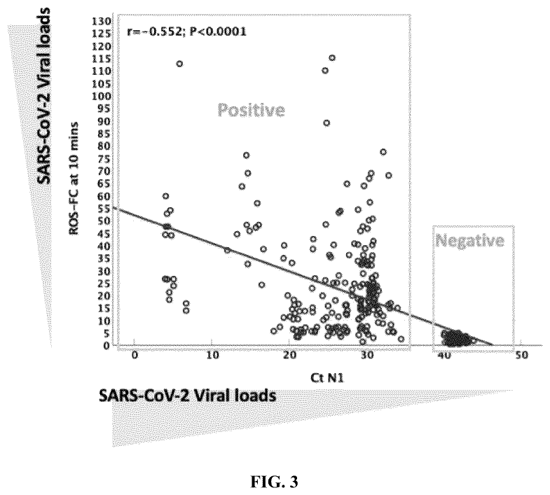

The ROS-FC₁ₘᵢₙ of tested nasopharyngeal swabs (n=375) when compared with Ct values of RT-qPCR for SARS-COV-2 showed significant correlations (FIGS. 2 and 3).

### 4. Validation of 96% Sensitivity and 100% Specificity Values of the Developed RBD-Aptasensor

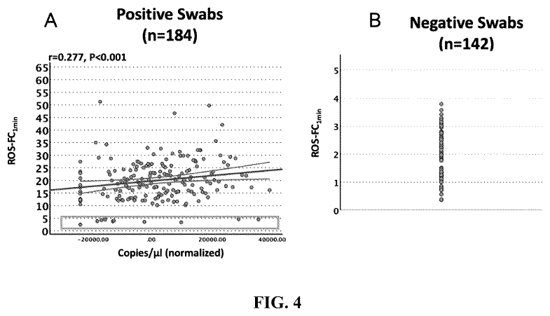

For the validation phase 326 nasopharyngeal swabs (184 positive and 142 negative RT-qPCR confirmed swabs) were selected. Out of 184 nasopharyngeal swabs that tested positive by RT-qPCR, 174 swabs were tested positive by RBD-aptasensor by having ROS more than 5 folds at 1 min (ROS-FC₁ₘᵢₙ≥5) of sample addition (97% sensitivity; Table 3 and FIG. 4A). The ROS-FC₁ₘᵢₙ values of nasopharyngeal swabs (n=326) showed significant correlations with the SARS-COV-2 viral loads of the tested swabs (FIG. 4A). Moreover, all the negative samples by RT-qPCR (n=100) showed less than 5 folds at 1 min (100% specificity: FIG. 4B).

TABLE 4
Overview of results of the clinical validation phase
of RBD-aptasensor for detection of SARS-CoV-2

| | RT-qPCR result | | |
|---|---|---|---|
| RBD-aptasensor result | Positive^{#} | Negative | Total |
| Positive* | 174 | 0 | 174 |
| Negative | 10 | 142 | 152 |
| Total | 184 | 142 | 326 |

*Positive result defined as ROS-FC_{1 min} ≥5.
^{#}Positive result defined as Ct < 40 of N1 gene of SARS-CoV-2.

Sequences

SEQ ID NO: 1 DNA sequence of the aptamer
ATCCAGAGTGACGCAGCATTTCATCGGGTCCAAAAGGGGCTGCTCGGGA
TTGCGGATATGGACACGT

What is claimed is:

1. A biosensor for detecting SARS-COV-2, comprising: an aptamer that can bind a protein, a fluorescent dye conjugated to the aptamer, and a graphene oxide solution, wherein the aptamer comprises the nucleotide sequence of SEQ ID NO: 1.

2. The biosensor of claim 1, wherein a fluorescent substance is conjugated to the nucleotide sequence of the aptamer.

3. The biosensor of claim 1, wherein the fluorescent substance comprises a fluorophore and the fluorophore comprises a 6-carboxyfluorescein.

4. The biosensor of claim 1, wherein the biosensor comprises a mixture of an approximately equal volume of graphene oxide and 6-carboxyfluorescein conjugated aptamer.

5. The biosensor of claim 1, wherein the aptamer can bind to antigens comprising the SARS-COV-2 virus.

6. The biosensor of claim 1, wherein the antigen comprises the SARS-COV-2 protein S.

7. The biosensor of claim 1, wherein the SARS-COV-2 protein S is detected in samples from patients with symptomatic or asymptomatic COVID-19.

8. The biosensor of claim 1, wherein the samples from patients are collected from nasopharyngeal swabs or from saliva samples.

9. The biosensor of claim 1, wherein the samples from patients are collected from nasopharyngeal swabs.

10. The biosensor of claim 1, wherein the samples are analyzed and the results are collected.

11. An aptamer-based method for detecting SARS-COV-2, comprising forming a biosensor, wherein the biosensor comprises an aptamer, a fluorescent substance conjugated to the aptamer, and graphene oxide, wherein a mixture of an approximately equal volume of graphene oxide and fluorescence conjugated aptamer is added to a detection area on a glass-based reading substrate.

12. The aptamer-based method of claim 11, wherein the fluorescent substance conjugated to the aptamer comprises a fluorophore.

13. The aptamer-based method of claim 11, wherein the fluorophore comprises a 6-carboxyfluorescein.

14. The aptamer-based method of claim 11, wherein the aptamer comprises the nucleotide sequence of SEQ ID NO: 1.

15. The aptamer-based method of claim 11, wherein the aptamer can bind to antigens comprising the SARS-COV-2 virus.

16. The aptamer-based method of claim 11, wherein the antigen comprises the SARS-COV-2 protein S.

17. The aptamer-based method of claim 11, wherein the SARS-COV-2 protein S is detected in samples from patients with symptomatic or asymptomatic COVID-19.

18. The aptamer-based method of claim 11, wherein the samples from patients are collected from nasopharyngeal swabs or from saliva samples.

19. The aptamer-based method of claim 11, wherein the samples from patients are collected from nasopharyngeal.

20. The aptamer-based method of claim 11, wherein the samples are analyzed and the results are collected.


SEQUENCE LISTING (1 sequence)

SEQ ID NO: 1 - DNA, 67 bp, synthetic construct
  source 1..67: mol_type = other DNA
atccagagtgacgcagcatttcatcgggtccaaaaggggctgctcgggattgcggatatggacacgt